(19) United States
(12) Patent Application Publication
(10) Pub. No.: US 2020/0104939 A1
(43) Pub. Date: Apr. 2, 2020

(54) METHODS OF ELECTRONICALLY MANAGING LIFE INSURANCE POLICY DETERMINATIONS, AND SYSTEMS AND NETWORKS FOR DOING THE SAME

(71) Applicant: The Penn Mutual Life Insurance Company, Horsham, PA (US)

(72) Inventors: David O'Malley, Ambler, PA (US); Gregory Driscoll, Philadelphia, PA (US); Raymond Caucci, Doylestown, PA (US); Andrew Martin, Chalfont, PA (US); Helen Rennie, Doylestown, PA (US); Gretchen Dinucci, West Hartford, CT (US); Mark Dash, Glenside, PA (US); Sheri Rhoades, Feasterville, PA (US)

(21) Appl. No.: 16/582,354

(22) Filed: Sep. 25, 2019

Related U.S. Application Data
(60) Provisional application No. 62/738,102, filed on Sep. 28, 2018.

(51) Int. Cl. G06Q 40/08; G06Q 40/02; G16H 50/30

(52) U.S. Cl. CPC G06Q 40/08; G16H 50/30; G06Q 40/025

(57) ABSTRACT

A method of determining whether to insure an applicant for life insurance is provided. The method includes the steps of: (a) electronically obtaining data from an applicant for life insurance, the data including personal data related to the applicant; (b) electronically underwriting a potential policy using the data obtained in step (a) and externally sourced data related to the applicant; and (c) electronically determining whether to insure the applicant after step (b), and if a determination is made to insure the applicant, electronically issuing an insurance policy for the applicant.

### CROSS REFERENCE TO RELATED APPLICATION

The application claims the benefit of U.S. Provisional Patent Application No. 62/738,102 filed on Sep. 28, 2018, the contents of which are incorporated herein by reference.

### FIELD

The invention relates to the management of life insurance policy determinations, and more particularly, to improved methods, systems, and networks for electronically managing certain life insurance policy determinations.

### BACKGROUND

Conventional life insurance application processes tend to be time consuming. An exemplary conventional process includes: (a) completing application paperwork; (b) scheduling, and participating in, a medical examination (e.g., typically in connection with a medical professional, such as a nurse); (c) an underwriting process conducted by an underwriter; (d) providing a rate to the applicant; as well as various other steps.

Various aspects of such conventional processes may involve paper submissions, human review, human interactions, etc. As such, conventional processes suffer from deficiencies such as human error and delay. Thus, it would be desirable to provide improved methods, systems, and networks for managing certain life insurance policy determinations.

### SUMMARY

According to an exemplary embodiment of the invention, a method of determining whether to insure an applicant for life insurance is provided. The method includes the steps of: (a) electronically obtaining data from an applicant for life insurance, the data including personal data related to the applicant; (b) electronically underwriting a potential policy using the data obtained in step (a) and externally sourced data related to the applicant; and (c) electronically determining whether to insure the applicant after step (b), and if a determination is made to insure the applicant, electronically issuing an insurance policy for the applicant.

According to another exemplary embodiment of the invention, another method of determining whether to insure an applicant for life insurance is provided. The method includes the steps of: (a) electronically initiating an application for life insurance by an adviser, the adviser inviting an applicant to register electronically; (b) registering electronically, by the applicant, to submit the application for life insurance; (c) electronically obtaining data from the applicant for life insurance, the data obtained from the applicant including personal data related to the application, at least a portion of the personal data being obtained from the applicant through use of a question driver, wherein the question driver prompts the applicant with a plurality of questions, and the question driver provides additional questions based on the applicant's answers to the plurality of questions; (d) electronically underwriting a potential policy using the data obtained in step (c) and externally sourced data related to the applicant, wherein the personal data is used to retrieve the externally sourced data; (e) electronically determining whether to insure the applicant after step (d); (f) electronically generating an electronic application package after step (e); (g) electronically signing, by the applicant, the electronic application package; (h) electronically submitting the electronic application package after step (g); (i) electronically issuing an insurance policy for the applicant; (j) electronically delivering the insurance policy to the applicant; (k) electronically signing, by the applicant, the insurance policy after step (j); (l) electronically providing payment, by the applicant, for at least a portion of the insurance policy; and (m) providing the client with access to information related to the insurance policy via an electronic client portal.

According to yet another exemplary embodiment of the invention, an insurance determination network for determining whether to insure an applicant for life insurance is provided. The insurance determination network includes a client device, a plurality of third party data providers (and associated devices), and an insurance determination system. The insurance determination system includes a data collector for receiving data from the client device and the plurality of third party data providers. The insurance determination system also includes a question driver for interacting with the client device to prompt an applicant to provide personal data to the system related to the applicant. The insurance determination system also includes an automated underwriting device utilizing (i) a rules engine and (ii) a predictive model to provide a risk rating for the applicant.

### BRIEF DESCRIPTION OF THE DRAWINGS

The invention is best understood from the following detailed description when read in connection with the accompanying drawings. It is emphasized that, according, to common practice, the various features of the drawings are not to scale. On the contrary, the dimensions of the various features are arbitrarily expanded or reduced for clarity. Included in the drawings are the following figures:

FIG. 1 is a flow diagram illustrating a method of determining whether to insure an applicant for life insurance in accordance with an exemplary embodiment of the invention;

FIG. 2 is a flow diagram illustrating another method of determining whether to insure an applicant for life insurance in accordance with another exemplary embodiment of the invention; and

FIG. 3 is a block diagram illustrating an insurance determination network for determining whether to insure an applicant for life insurance in accordance with yet another exemplary embodiment of the invention.

### DETAILED DESCRIPTION

In accordance with certain exemplary embodiments of the invention, a digital life insurance purchasing process is enabled. That is, most (and in some cases all) process steps associated with the purchase of life insurance are electronic in nature, thus improving the quality of the purchasing experience for both the vendor (e.g., the life insurance company), the adviser, and the applicant (i.e., the potential client). For many prospective clients, such a process may avoid paramedical exams (i.e., parameds), other medical screenings, and paper document preparation and execution For vendors, such a process may provide benefits such as enabling working with smaller scale clients, and improved accuracy in the process with reduced opportunities for error. For the advisers, increased and faster sales may be provided, with reduced time to receive a commission.

According to aspects of the invention, an end-to-end digital experience is provided. An electronic application (i.e., “e-app”) is provided for use by the adviser and the applicant. Electronic delivery (i.e., “e-delivery”) of documents is provided, and electronic payment (i.e., “e-pay”) by the applicant is also enabled. Further still, electronic service (i.e., “e-service”) is provided by the vendor and adviser to the applicant (later, the client, after the policy is in force), for example, through a client portal.

Aspects of the invention significantly reduce the cycle time of providing a life insurance policy. For example, in some cases, the policy may be provided to the applicant from start to finish in a matter of one hour (and in some cases, even less). Of course, such timing requires active participation, for example, from the adviser and applicant (e.g., to respond to queries from the e-app, to e-sign documents in a timely manner, to make electronic payments in a timely manner, etc.).

Further, the process (and related systems) allows applicants (and advisers) to interact with the system (including the system applications) through a mobile device such as a smart phone, tablet, etc., or any other computer device.

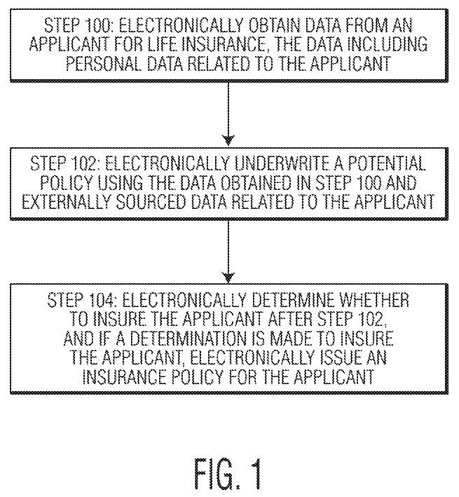

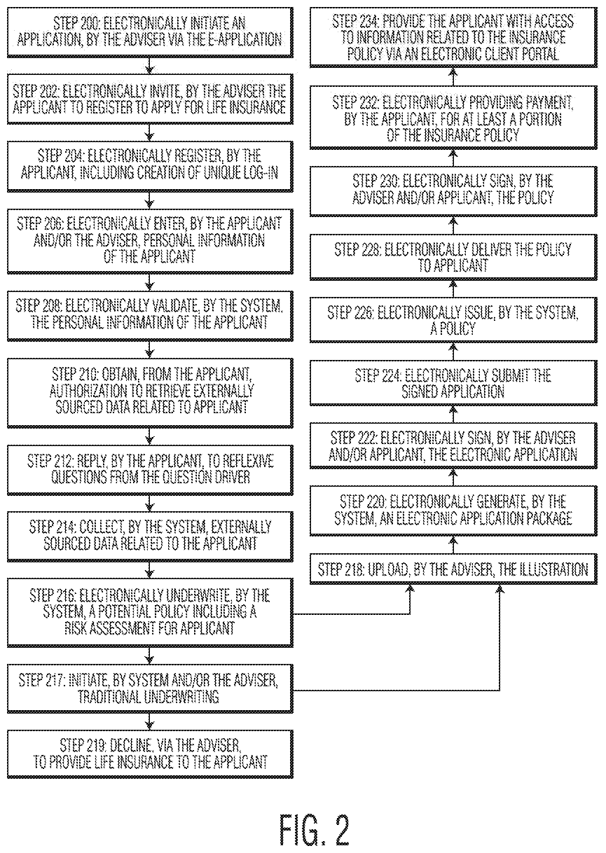

FIGS. 1-2 are flow diagrams in accordance with certain exemplary embodiments of the invention. As is understood by those skilled in the art, certain steps included in the flow diagrams may be omitted; certain additional steps may be added; and the order of the steps may be altered from the order illustrated.

As will be appreciated by those skilled in the art, FIG. 1 is a more generic exemplary process, while FIG. 2 is a much more specific exemplary process. Any of the steps of FIG. 2, as well as additional steps, may be incorporated into the method of FIG. 1.

FIG. 1 is a flow diagram illustrating a method of determining whether to insure an applicant for life insurance. At Step 100, data is electronically obtained from an applicant for life insurance, where the data includes personal data related to the applicant. Such data may include personal identification data (e.g., name, social security number, date of birth, address, etc.) as well as medical data (e.g., active medical conditions, prior medical conditions, family history, blood pressure, weight, etc). Some of this data may be provided by an adviser. However, the data is largely provided by the applicant through a “question driver” process/algorithm enabled by the system. The question driver prompts the applicant with a plurality of questions, and the question driver provides additional questions based on the applicant's answers to the plurality of questions. At Step 102, a potential policy is electronically underwritten using the data obtained in Step 100 and externally sourced data related to the applicant. For example, the externally sourced data may be provided by the system through interactions with third party sources, using the personal data provided by the applicant and/or the adviser. Such externally sourced data may include, for example, medical history information, driving records, prescription drug use, etc.). Using the personal data provided by the applicant (and/or adviser), and the externally sourced data, a potential life insurance policy is electronically underwritten. Such an electronic underwriting may include, for example, the assignment of a rating to the applicant. At Step 104, a determination is electronically made as to whether to insure the applicant after Step 102. If a determination is made to insure the applicant, a life insurance policy is electronically provided to the applicant. For example, if the rating (or other criteria from the electronic underwriting process) is favorable, a life insurance policy may be electronically offered to the applicant. Otherwise, the adviser may be notified electronically that a policy will not be offered, or that traditional underwriting will need to review the application.

FIG. 2 is a flow diagram illustrating another method of determining whether to insure an applicant for life insurance. At Step 200, an application for life insurance is electronically initiated by an adviser. Specifically, the adviser initiates a digital process using an electronic application (i.e., the e-app). For example, the adviser uses their personal device (e.g., a computer, a tablet, a smart phone, etc.) to log in to the e-app, and creates an application for the applicant. In connection with this process initiation by the adviser, at Step 202, the adviser invites the applicant (e.g., via email with a link) to register electronically (i.e., to register to apply for life insurance). At Step 204, the applicant electronically registers with the system so that an application may be submitted for life insurance. In connection with the applicant registration, the applicant may be prompted to create a unique client identifier (e.g., a unique log-in) such as a user name, password, or other log-in criteria. At Step 206, personal information (also referred to as personal data such as personal identification data and medical data related to the applicant) is obtained by the system. Some of this data may be provided by the adviser, but most of this data is provided by the applicant electronically through use of a question driver. The question driver prompts the applicant with a plurality of questions, and the question driver provides additional questions based on the applicant's answers to the plurality of questions. At Step 208, the system electronically validates the personal information of the applicant. At Step 210, authorization is obtained, from the applicant, to retrieve externally sourced data related to the applicant. At Step 212, the applicant replies to reflexive questions from the question driver (this step may be done in conjunction with, or in addition to, the collection of personal information in Step 206). At Step 214, the system collects the externally sourced data authorized by the applicant in Step 210. At Step 216, the system electronically underwrites a potential life insurance policy for the applicant including a risk assessment. At Step 218, if the rating (and/or risk assessment) determined at Step 216 is acceptable, the adviser is prompted, and the adviser then uploads the illustration (an insurance profile, perhaps including predictive modeling, understood by those skilled in the art).

If the rating (and/or risk assessment) determined at Step 216 is not acceptable, at Step 217 the system and/or the adviser initiates traditional underwriting (e.g., involving a human underwriter). At Step 219, after the traditional underwriting, if the rating (and/or other risk assessment) is still not acceptable, the adviser instructs the applicant that the policy has been declined. At Step 219, after the traditional underwriting, if the rating (and/or other risk assessment) is now deemed acceptable, the process proceeds to Step 218, and the illustration is uploaded by the adviser.

Following Step 218, at Step 220, an electronic application package is electronically generated by the system. At Step 222, the adviser and/or the applicant electronically signs the electronic application. Typically, the adviser would first electronically sign the application, and then the applicant would be prompted to electronically sign the application. At Step 224, the signed application is electronically submitted. At Step 226, a life insurance policy for the applicant is electronically issued by the system. At Step 228, the life insurance policy is electronically delivered (e.g., via email) to the applicant. At Step 230, the adviser and/or the applicant electronically signs the policy. Typically, the adviser would first electronically sign the application, and then the policy would be delivered to the applicant for electronic signature. At Step 232, the applicant electronically provides payment for the policy (or at least a portion of the policy, as required). At Step 234, the applicant is provided with access to information related to the insurance policy (e.g., including a copy of the policy itself) via an electronic client portal. Thus, as illustrated above in connection with the description of FIG. 2, an end-to end digital process is enabled for the acquisition of a life insurance policy.

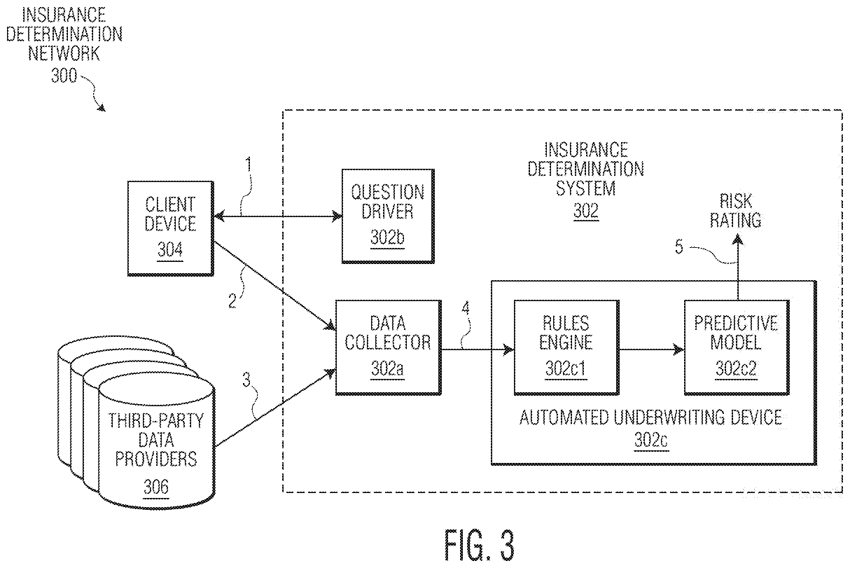

The invention may also be embodied as a “system” for determining whether to insure an applicant for life insurance. FIG. 3 is a block diagram illustrating an example of such a system. More specifically, FIG. 3 illustrates an insurance determination network 300 including an insurance determination system 302 (herein insurance determination system 302 is an example of what is referred to throughout the present application simply as, the “system”). Insurance determination network 300 also includes a client device 304, and third party data providers 306 (and their related devices).

Insurance determination system 302 includes data collector 302a (including hardware and software), question driver 302b, and automated underwriting device 302c. Automated underwriting device 302c includes rules engine 302c1 and predictive model 302c2. Not shown in FIG. 3 is the adviser device(s), it being understood that such a device(s) may be integrated into insurance determination system 302, may be a separate device(s) of the adviser, or both.

An interaction of the elements of FIG. 3 is now provided, referring to interaction numbers “1” through “5” shown in FIG. 3. Interaction “1” illustrates client device 304 interoperating with question driver 302b to receive queries, for example, the applicant medical questions. These questions may be reflexive in that additional questions are asked based on previous answers. At interaction “2”, the client device 304 transmits applicant questions and answers to data collector 302a. Data collector 302a interoperates with third-party providers 306 to collect additional data related to the applicant, as shown at interaction “3”. Such data is Fair Credit Reporting Act compliant. Example third-party data providers include: State Department of Motor Vehicle driving history report providers, Medical Information Bureau insurance report providers, Risk Score report providers, prescription drug history report providers, among others. At interaction “4”, the data from data collector 302a is transmitted to the automated underwriting device 302c. Rules Engine 302c1, and Predictive Model 302c2, are used to generate a risk rating for the applicant as shown at interaction “5”. As will be appreciated by those skilled in the art, the hardware and/or software associated with the various elements of insurance determination system 302 may be combined into one or more physical elements, and/or one or more software systems.

Although the invention is illustrated and described herein with reference to specific embodiments, the invention is not intended to be limited to the details shown. Rather, various modifications may be made in the details within the scope and range of equivalents of the claims and without departing from the invention.

What is claimed:

1. A method of determining whether to insure an applicant for life insurance, the method comprising the steps of:
(a) electronically obtaining data from an applicant for life insurance, the data including personal data related to the applicant;
(b) electronically underwriting a potential policy using the data obtained in step (a) and externally sourced data related to the applicant; and
(c) electronically determining whether to insure the applicant after step (b), and if a determination is made to insure the applicant, electronically issuing an insurance policy for the applicant.

2. The method of claim 1 further comprising a step of initiating an application for life insurance by an adviser before step (a), the adviser inviting the applicant to register electronically prior to step (a).

3. The method of claim 2 further comprising a step of registering electronically, by the applicant, to submit the application for life insurance before step (a).

4. The method of claim 3 wherein the step of registering includes creating, by the applicant, a unique log-in for the applicant.

5. The method of claim 1 wherein step (a) includes receiving the data from the applicant through use of a question driver.

6. The method of claim 5 wherein the question driver prompts the applicant with a plurality of questions, and the question driver provides additional questions based on the applicant's answers to the plurality of questions.

7. The method of claim 1 further comprising the step of electronically obtaining authorization from the applicant to retrieve the externally sourced data.

8. The method of claim 1 wherein the personal data is used to retrieve the externally sourced data utilized in step (b).

9. The method of claim 1 wherein the externally sourced data includes at least one of medical data related to the applicant, prescription data related to the applicant, and credit data related to the applicant.

10. The method of claim 1 wherein after at least one of step (a) and step (b), determining that the application requires traditional underwriting, whereby the traditional underwriting is used in connection with step (c).

11. The method of claim 1 further comprising the step of (d) electronically generating an electronic application package after step (c).

12. The method of claim 11 further comprising the step of (e) electronically signing, by the applicant, the electronic application package.

13. The method of claim 12 further comprising the step of (f) electronically submitting the electronic application package after step (e).

14. The method of claim 13 further comprising the step of (g) electronically issuing an insurance policy for the applicant.

15. The method of claim 14 further comprising the step of (h) electronically delivering the insurance policy to the applicant.

16. The method of claim 15 further comprising the step of electronically signing, by the applicant, the insurance policy after step (h).

17. The method of claim 16 further comprising the step of (j) electronically providing payment, by the applicant, for at least a portion of the insurance policy.

18. The method of claim 17 further comprising the step of (k) providing the client with access to information related to the insurance policy via an electronic client portal.

19. A method of determining whether to insure an applicant for life insurance, the method comprising the steps of:
(a) electronically initiating an application for life insurance by an adviser, the adviser inviting an applicant to register electronically;
(b) registering electronically, by the applicant, to submit the application for life insurance;
(c) electronically obtaining data from the applicant for life insurance, the data obtained from the applicant including personal data related to the application, at least a portion of the personal data being obtained from the applicant through use of a question driver, wherein the question driver prompts the applicant with a plurality of questions, and the question driver provides additional questions based on the applicant's answers to the plurality of questions;
(d) electronically underwriting a potential policy using the data obtained in step (c) and externally sourced data related to the applicant, wherein the personal data is used to retrieve the externally sourced data;
(e) electronically determining whether to insure the applicant after step (d);
(f) electronically generating an electronic application package after step (e);
(g) electronically signing, by the applicant, the electronic application package;
(h) electronically submitting the electronic application package after step (g);
(i) electronically issuing an insurance policy for the applicant;
(j) electronically delivering the insurance policy to the applicant;
(k) electronically signing, by the applicant, the insurance policy after step (j);
(l) electronically providing payment, by the applicant, for at least a portion of the insurance policy; and
(m) providing the client with access to information related to the insurance policy via an electronic client portal.

20. An insurance determination network comprising:
(a) a client device;
(b) a plurality of third party data providers; and
(c) an insurance determination system,
the insurance determination system including a data collector for receiving data from the client device and the plurality of third party data providers,
the insurance determination system also including a question driver for interacting with the client device to prompt an applicant to provide personal data to the system related to the applicant,
the insurance determination system also including an automated underwriting device utilizing (i) a rules engine and (ii) a predictive model to provide a risk rating for the applicant.
